(19) United States
(12) Patent Application Publication
(10) Pub. No.: US 2016/0058434 A1
(43) Pub. Date: Mar. 3, 2016

(54) Closure Device and Methods and Systems for Using Same

(71) Applicant: Symetis SA, Ecublens (CH)

(72) Inventors: Stephane Delaloye, Bulach (CH); Jean-Luc Hefti, Cheseaux-Noreaz (CH); Reynald Passerini, Lausanne (CH)

(21) Appl. No.: 14/936,168

(22) Filed: Nov. 9, 2015

Related U.S. Application Data
(63) Continuation of application No. 14/314,819, filed on Jun. 25, 2014, now abandoned.
Application No. 14/314,819 is a continuation of application No. 13/375,825, filed on Dec. 2, 2011, now abandoned.
(60) Provisional application No. 61/183,791, filed on Jun. 3, 2009.

(51) Int. Cl. A61B 17/00

(52) U.S. Cl. CPC A61B 17/0057; A61B 2017/00575; A61B 2017/00597; A61B 2017/0061; A61B 2017/00623; A61B 2017/00628; A61B 2017/00867

(57) ABSTRACT

Embodiments of the present disclosure are directed to apical closure systems, devices, and methods and systems for use thereof, for closing surgical openings or defects in the wall of the heart.

### FIELD OF THE DISCLOSURE

Embodiments of the present disclosure are directed to closure devices, more particularly, apical closure devices and methods and systems for use thereof, for closing surgical openings in the wall of the heart (for example).

### BACKGROUND OF THE DISCLOSURE

Cardiovascular disease or cardiovascular diseases refers to the class of diseases that involve the heart or blood vessels (arteries and veins). This class of disease thus refers to any disease that affects the cardiovascular system any may include atherosclerosis (arterial disease), coronary artery disease, valvular heart disease, ischemic heart disease (IHD), or myocardial ischaemia. These diseases are characterized by reduced blood supply to the heart muscle, usually due to coronary artery disease (atherosclerosis of the coronary arteries). Depending on the symptoms and risk, treatment may be with medication, percutaneous coronary intervention (angioplasty) or conventional open-heart surgery.

Best known of the current techniques for the treatment of severe cardiovascular disease is conventional open-heart surgery, which may be used to perform coronary artery bypass grafting, heart valve repair or replacement, etc. Coronary artery bypass grafting is a relatively invasive technique wherein a thoracotomy is performed to expose the patient's heart, and one or more coronary arteries are bypassed with arteries or veins from elsewhere in the patient's body, or with synthetic grafts. Valve replacement is a cardiac surgery procedure in which a patient's heart valve is replaced by a prosthetic valve. Heart valve replacement therapy typically performed when the valve becomes too tight (valvular stenosis) for blood to flow across the valve, or too loose (valvular incompetence) in which case blood can leak into the reverse direction. Some individuals may have a combination of valve stenosis and valve incompetence (valvular steno-insufficiency) or simply one or the other. In some cases, in addition, the valve malfunctioning may affect more than one heart valve at the same time.

Conventional approaches for cardiac valve replacement require the surgical cutting of a relatively large opening in the patient's sternum (“sternotomy”) or thorax (“thoracotomy”) in order to allow the surgeon to access the patient's heart. For example, conventional open-heart surgery or cardiac valve replacement is most frequently done through a median sternotomy, meaning the chestbone is sawed in half. Once the pericardium has been opened, the patient is placed on cardiopulmonary bypass machine, also referred to as the heart-lung machine. This machine takes over the task of breathing for the patient and pumping their blood around while the surgeon replaces the heart valve. Additionally, these approaches require arrest of the patient's heart. These techniques are thus extremely invasive and are accompanied risk of death or serious complications, depending on the comorbidities and age of the patient. Older patients, as well as more fragile ones, are sometimes ineligible for surgery because of elevated risks.

In recent years, efforts have been made to establish a less invasive cardiac valve replacement procedure, by delivering and implanting a cardiac prosthetic valve via a catheter (i.e., transcatheter procedure) via either a transfemoral approach—delivering the new valve through the femoral artery, or by a transapical route, where the prosthetic valve is delivered between ribs and directly through the wall of the heart to the implantation site.

While less invasive and arguably less complicated, percutaneous heart valve replacement therapies (PHVT) still have various shortcomings, including the difficulty for the implanter to ensure proper positioning of the prosthetic valve within the patient's body. Specifically, if the replacement valve is not placed in the proper position relative to the implantation site, it can affect the safety and efficacy of the valve. For example, in an aortic valve replacement, if the replacement valve is placed too high, it can lead to valve regurgitation, instability, coronary occlusion. If the valve is placed too low, it can also lead to regurgitation, to AV/block and interference with the mitral valve.

Off-pump trans-left ventricular approach (transapical approach) provides a more precise and reliable deployment of transcatheter aortic valve of any size compared to the peripheral (transfemoral) procedure. See e.g., Tozzi et al., Eur J Cardiothorac Surg. 2007 January;31(1):22-5. As mentioned above, a transventricular approach involves creating an access opening (access aperture in the free wall of the ventricle, through which the prosthetic valve is implanted using a catheter. One of the key steps of the transventricular approach, however, is the closure of the ventricular access opening after the prosthetic valve has been implanted. Safely closing a surgical opening of the free wall of the ventricle can be a challenging procedure, even because it must be performed without any support of extracorporeal circulation. Further, fragile tissues may lead to technical difficulties, especially when closing larger holes while being off-pump in high-risk elderly patients. The conventional technique involves a surgical step of placing purse-string sutures around the access opening. Tensioning and tying off the sutures seals the aperture. However, the conventional technique involves surgical access to the heart, which may result in the transventricular procedure remaining more invasive than the peripheral (transfemoral) procedure.

WO-A-2007/071436 proposes an alternative technique using a guidewire-compatible occluder device for sealing the ventricular access opening following implantation of a prosthetic valve. The occluder device comprises a stent-like component having opposed first and second oversize cones that define a tapered waist or groove between the cones. The occluder is delivered to the access opening such that one cone expands on the ventricular side of the heart wall, and the other cone expands on the outside of the heart wall. The opposed oversize cones sandwich the heart wall resiliently from either side to retain the occluder in position and occlude the access opening to seal it. While this technique is said to achieve a reliable and tight fit, and offers the potential of delivering the occluder along a guidewire to the access opening, and in particular along the same guide wire used for implantation of the prosthetic valve, the occluder has not been adopted. The oversize cones have inherently large profiles inside and outside the heart.

Thus, despite the progress made in the development of trans-apical aortic valve implant, closure of the access opening in the heart remains a major issue. Accordingly, there remains a need for devices and methods that can aid the in closure of a ventricle opening following procedures to treat cardiovascular disease.

Throughout this description, including the foregoing description of related art, any and all publicly available documents described herein, including any and all U.S. patents, are specifically incorporated by reference herein in their entirety. The foregoing description of related art is not intended in any way as an admission that any of the documents described therein, including pending United States patent applications, are prior art to embodiments of the present disclosure. Moreover, the description herein of any disadvantages associated with the described products, methods, and/or apparatus, is not intended to limit the disclosed embodiments. Indeed, embodiments of the present disclosure may include certain features of the described products, methods, and/or apparatus without suffering from their described disadvantages.

### SUMMARY OF THE DISCLOSURE

The present disclosure provides embodiments for systems, devices and methods for the closure of any passage through the wall of a heart, and in particular, through the ventricle wall for any transcatheter procedure. Accordingly, for some embodiments, the present disclosure provides for devices and methods for ventricular apical closure.

The present invention provides for a Left Ventricle (LV) trans-apical procedure that has been developed for transcatheter aortic implants. The LV trans-apical access procedures may be conveniently used for the transcatheter treatment of mitral valve (both valve implant and valve repair).

Further, the procedures and devices of the present invention may be used in conjunction with procedures dealing with the left side of the heart, which may require a trans-wall/trans-apical access (e.g., cordae tendinae repair or replacement, etc.). In all these cases, the access closure device of the present invention may be used. Such use will help to speed the procedure and mitigate the risk of adverse events.

The devices and trans-wall procedures of embodiments the present invention can be used to treat the valves positioned in the right side of the heart. Right Ventricle (RV) trans-wall/trans-apical access to the pulmonary and to the tricuspid valve have identical characteristics as in the case of LV. Hence, the closure device is compatible also with the RV access.

According to some embodiments, systems, devices and methods for the closure of any passage through the ventricle wall for any transcatheter procedure are provided.

According to some embodiments, systems, devices and methods for left ventricular apical closure in a system using minimally invasive transapical transcatheter techniques for valve prosthesis implant are provided.

According to some embodiments, the devices of the present disclosure comprise the following three components: (1) a support structure, preferably capable to be deformed for the positioning (reducing outer diameter, recovering a substantial cylindrical shape) and to self-adapt to the characteristics of the implanting site; (2) cover component covering partially or totally the outer surface of the support structure; and (3) a plug for filling a cavity or lumen of the support structure and capable to be punctured and passed through by a guidewire. At least some of the embodiments of the present disclosure allow for both the positioning of the closure device over the wire and the reintroduction of a guidewire in the ventricle at any moment after the closure of a trans-wall opening, e.g., a ventricular opening established to access the interior of the heart.

According to some embodiments, a closure device includes at least one and preferably all of the following components: a support structure, preferably capable to be deformed for the positioning (reducing outer diameter, recovering a cylindrical shape) and possibly to self-adapt to the geometry of the opening; biological tissues and/or artificial fabrics covering partially or totally the outer surface of the support structure; and a plug, and/or filling material for filling the cavity and capable to be punctured and passed through by a guidewire.

In some embodiments, systems, devices and methods are provided which enable the positioning of a closure device over a guidewire. Optionally, reintroduction of a guidewire in the ventricle may additionally be enabled at any moment after the closure of the ventricular access opening. Alternatively, the closure device may be configured not to facilitate reintroduction of a guidewire.

According to some embodiments, a closure device is provided which includes at least one and preferably one or more, and most preferably, all of the following: a meshed metallic support structure, preferably capable to be deformed for the positioning (reducing outer diameter, recovering a cylindrical shape); biological tissues and/or artificial fabrics covering partially or totally the outer surface of the support structure; and a plug and/or filling material for filling the cavity and capable to be punctured and passed through by a guidewire. Some of the embodiments of the present disclosure may allow for both the positioning of the device over the wire and the reintroduction of a guidewire in the ventricle at any moment after the closure of the ventricular opening/surgical opening.

Some embodiments of the present disclosure provide for a device for closing an opening or defect in a cardiac wall of a heart and may comprise at least one of, and preferably one or more of, and most preferably all of: a support structure; a cover component covering partially or totally the outer surface of the support structure; and a plug (and/or filling material) for filling the interior lumen of the support structure. Preferably, the support structure is a meshed metallic support structure. More preferably, the support structure is a self-expandable meshed metallic support structure.

Some embodiments of the present disclosure provide for a device for closing an access opening in the wall of a heart (e.g., ventricle wall), and may comprise at least one of, and preferably one or more of, and most preferably all of: a support structure; a cover component covering partially or totally the outer surface of the support structure; and a plug for filling the interior lumen of the support structure. Preferably, the support structure is a meshed metallic support structure. More preferably, the support structure is a self-expandable meshed metallic support structure.

According to some embodiments described herein, the support structure comprises a flange portion (which may be comprised of a plurality of “spokes”, “petals”, or “blades”, each of the foregoing terms being used interchangeably in the present disclosure), a cylindrical portion, and an optional outwardly expanding conical portion. According to some embodiments described herein, the diameter of the flange section may be between about 16 mm to about 40 mm.

The flange portion may be formed by bending of a cylindrical wire form, such that, during expansion of the device, the flange portion expands and is finally positioned after expansion into a form which forms and angle with the cylindrical portion of the device. In some embodiments, the angle is between about 0 and 45 degrees; in other embodiments between about 45 degreees and 90 degrees, in other embodiments of between about 90 degrees and 120 degrees, and in other embodiments between about 120 degrees and 180 degreees.

According to some embodiments described herein, the diameter of the cylindrical portion preferably exceeds a trans-wall access opening diameter.

According to some embodiments described herein, the diameter of the cylindrical portion may be between about 8 mm to about 18 mm.

According to some embodiments described herein, the length of the cylindrical portion may be between about 10 mm to about 25 mm.

According to some embodiments described herein, the length of the cylindrical portion and the optional outwardly expanding conical portion may be between about 10 mm to about 25 mm.

According to some embodiments described herein, the length of the cylindrical portion may be from about 6 to about 16 mm and the length of the outwardly expanding conical portion may be from about 4 mm to about 10 mm.

According to some embodiments described herein, the angle that defines the outward deflection of an optional third conical portion of the support structure may be between about 0° to about 20°.

According to some embodiments described herein, the closure device may further comprise one or more, and preferably a plurality of protrusions capable of anchoring the closure device in the ventricular wall. The protrusions may be provided for on at least one end of the closure device. In some embodiments, the protrusion comprises bent portions of the mesh that makes up an end of the closure device. The protrusion may protrude outwardly at an angle, and in some embodiments, such an outward angle directs the end of the protrusion back toward the opposite end of the closure device. The angle may be between about 5 degrees and about 90 degrees relative to the center longitudinal axis of the closure device, and more preferably, between about 30 and about 50 degrees relative the center longitudinal axis of the closure device.

According to some embodiments described herein, the support structure may be capable of being deformed for positioning across the ventricular wall.

According to some embodiments described herein, the cover component may be made up of a material that is at least one of flexible, compressible, host-compatible, and non-thrombogenic.

According to some embodiments described herein, the cover component may be made up of any of natural biocompatible materials, synthetic biocompatible materials, or combinations of mixtures thereof.

According to some embodiments of the invention, the cover component may be made of mammal pericardium.

According to some embodiments described herein, the cover component may cover at least 50% of the outer surface of the support structure

According to some embodiments described herein, the plug (which may also be referred to as a filling material, or a filling material may be used in combination with a plug material) may be capable of being punctured and passed through by a guidewire.

According to some embodiments described herein, the plug may be a polymeric plug, a foldable haemostatic valve, foam, a polymeric sponge filling and/or a combination thereof.

According to some embodiments described herein, the plug may be a self-sealing silicone plug that fills the inner lumen of the support structure.

Accordingly, the present disclosure provides for systems, devices and methods for sealing ventricular ports, surgical openings or defects. In some embodiments, methods may include delivering an implantable expandable closure device into the ventricle via a catheter, expanding the device to assume the size and shape of the opening, port or defect in the wall of the left ventricle, thereby sealing the port, opening or defect in the wall of the ventricle. The methods may further comprise anchoring the closure device in the ventricular wall.

In some embodiments, upon delivery and expansion of the closure device in the opening for occluding, the interior of the closure device for housing the plug and/or filling material, receives the plug and/or filling material only after expansion of the closure device. In still other embodiments, the plug and/or filling material may be present in the interior of the closure device when the closure device is in a compressed/unexpanded position.

According to some embodiments, a ventricle closure device is provided that is able to adapt itself not only to the diameter of the surgical hole or other defect (radial expansion to accommodate or fit the diameter of the “hole”), but also to the ventricular wall thickness. Thus, according to some embodiments, the closure device adapts its axial length to the wall thickness. Examples of such embodiments, are described in FIG. 4 and FIG. 5. The solutions exemplified in these embodiments aim is to embrace the ventricular wall with the support structure (e.g., support metallic structure).

According to some embodiments, a ventricle closure is provided having a support structure that is collapsible to a delivery state for implantation via an access opening in a ventricle wall, and self-expandable to a deployed or operative state upon implantation. The support structure may comprise a first portion for positioning or fitting in the access opening and/or a flange portion for being arranged substantially flat against the ventricle wall to prevent migration of the closure device in at least one direction. The flange portion may be for a distal portion of the ventricle closure fitting inside the ventricle and/or for a proximal portion of the ventricle closure fitting externally of the ventricle. Use of a flange portion fitting substantially flat against the ventricle wall can provide a low profile that does not project substantially with respect to the surface of the ventricle wall. If used inside the ventricle, the low profile is advantageous in reducing any risk of damage to internal heart tissue, and reducing possible interference to blood flow within the ventricle. If used outside the ventricle, the low profile is advantageous in reducing any risk of damage to the body surrounding the heart, for example, pericardium tissue. The ventricle closure may further comprise a cover component covering at least partially the outer surface of the support structure.

According to some embodiments, a ventricle closure is provided having a support structure that is collapsible to a delivery state for implantation via an access opening in a ventricle wall, and self-expandable to a deployed or operative state upon implantation. The support structure may comprise a first portion for positioning or fitting in the access opening and/or an anchor portion configured for bearing against the ventricle wall to prevent migration of the closure in at least one direction following implantation. The support structure may be configured not to project substantially from the exterior surface of the ventricle wall (e.g., from between 0 mm to about 2 mm, and in some embodiments, between about 0.001 mm and about 2 mm). The support structure thus has a low profile height on the external side, reducing any risk of damage to the body surrounding the heart, for example, pericardium tissue. The ventricle closure may further comprise a cover component covering at least partially the outer surface of the support structure.

In some embodiments, a ventricle closure is provided having a support structure that is collapsible to a delivery state for implantation via an access opening in a ventricle wall, and self-expandable to a deployed or operative state upon implantation. The support structure may comprise a first portion for positioning or for fitting in the access opening and/or an anchor portion configured for bearing against the ventricle wall to prevent migration of the closure in at least one direction following implantation. The anchor portion may comprise a plurality of radially outwardly projecting independent blades. Use of independent blades may enhance the conformability of the anchor portion to adapt to an asymmetric ventricle wall contour. For example, near the apex of the heart, the ventricle wall is highly concavely curved. A portion of the anchor to one side of access opening may need to adapt to a contour different from a portion of the same anchor diametrically opposite. One portion may be highly curved, and another portion of the same anchor relatively flat. Use of a plurality of independent blades may provide greater conformability than, for example, an anchor portion having an outer support ring circumscribing the anchor portion. Use of independent blades may also facilitate collapse of a relatively large anchor portion to a relatively compact form for implantation, because the blades can be flexed independently without one blade resisting flexing of an adjacent blade. Optionally, each independent blade may extend from the first portion. Optionally, each blade may comprise first and second struts coupled at their tips remote from the first portion. The ventricle closure may further comprise a cover component covering at least partially the outer surface of the support structure.

In the aforementioned definitions, the first portion of the support structure may be tubular. The first portion may optionally be a hub carrying the flange and/or anchor portion. The first portion may optionally have a shape selected from cylindrical, conical, frustum, tapered, flared. Alternatively, the first portion may optionally comprise plural regions of different shape. At least one shape region, and preferably at least two, may be selected from the shapes of cylindrical, conical, frustum, flared, tapered. The first portion may be referred to as a spigot portion for spigot-socket relation with, or positioning within, the access opening.

In some embodiments, a delivery apparatus for delivering a ventricle closure to an access opening may comprise: a sheath for at least partly surrounding the closure device and defining a compartment to constrain the closure device to a delivery state in which the closure device is collapsed for delivery and implantation; and/or a guidewire lumen coupled to the sheath and positioned externally of the compartment for the closure device. Use of a guidewire lumen external to the compartment may facilitate delivery over a guidewire without the guidewire having to pass through the closure device itself. An advantage is that the closure device may avoid a self-closing valve or material for closing the guidewire aperture.

In some embodiments, an assembly or combination may be provided comprising a closure device and a delivery device. The closure device may be implantable via an access opening in the ventricle wall of a heart and operative to close the access opening. The delivery device may be for delivering the closure device to access opening. The closure device may be collapsible to a delivery state for implantation via the access opening. In the delivery state, a distal end of the closure device may have a tapered tip. The distal end (at least) of the closure device may be self-expandable upon implantation. The delivery device may comprises a sheath surrounding a non-distal portion of the closure device to hold the delivery device in its delivery state, the tapered tip at the distal end being exposed and protruding from the sheath. Optionally, the distal end forms, when expanded, an anchor portion. Optionally, the distal end comprises a plurality of independent blades.

### BRIEF DESCRIPTION OF THE DRAWINGS

For a better understanding of the present invention, reference is made to the following description, taken in conjunction with the accompanying drawings, in which like reference characters refer to like parts throughout.

FIG. 1 provides a graphic of a human heart with an apical closure device according to some embodiments of the present disclosure is deployed in the wall of the left ventricle.

FIGS. 2 A-D show the shape and dimensions of a metallic support structure for a closure device according to some embodiments of the invention. FIG. 3 shows the shape of a metallic support structure having protrusions extending downward at an angle α2 according to some embodiments of the invention.

FIG. 4 provides a graphic of a closure device according to some embodiments of the present disclosure. The proximal (in respect to the operator) section of the stent (the portion of the structure adjacent section 260 as shown in FIGS. 2B-2C) has the capability to self-expand outwards if not constrained by the ventricular wall. The curvature and the rigidity of this proximal section may be chosen so that the outer edge of the structure always lays on the external surface of the ventricle. Hence, the radial stiffness of the mesh will decrease moving from distal to proximal section (e.g., reducing the radial thickness of the structure).

FIG. 5 shows a flange that is created in correspondence of the proximal edge of the stent (as noted above, the proximal edge being adjacent section 260). The outer diameter of this flange can be smaller than the diameter of the flange positioned within the ventricle. Axial elastic joint (compliant coils) make the length of the device (i.e., the distance between the two flanges) variable and self-adaptable to the wall thickness.

FIGS. 6A-B show a closure device including a cover component over a support structure. FIG. 6A is a schematic side view, and FIG. 6B is an end view from within the ventricle. The drawings omit detail of a proximal anchor portion in order to avoid clutter, but it will be appreciated that the configurations of any of FIGS. 2 to 5 may be implemented as desired. FIG. 6C is a schematic perspective view of an optional plug for fitting within the lumen of the support structure.

FIGS. 7A-B show a first example of delivery device loaded with a closure device in a delivery state. The closure device is shown by the form of the support structure (the cover component and optional plug are omitted to avoid obscuring other features). The delivery device includes a sheath at least partly surrounding the closure device, and defining a compartment for constraining the closure device to its delivery state. A guidewire lumen passes through the compartment, to enable the delivery device to be guided to an access opening for implanting the delivery device. FIG. 7A is a schematic side section, and FIG. 7B is an end view of the tip.

FIGS. 8A-B show a second example of a delivery device loaded with a closure device, similar to FIG. 7. The principle difference is that the sheath does not surround a distal end of the closure device. Instead, the distal end of the closure device is exposed.

FIGS. 9A-B show a third example of a delivery device loaded with a closure device, similar to FIG. 7. The principle difference is that the guidewire lumen is disposed outside the compartment containing the closure device. The guidewire lumen may be coupled to the sheath outside the compartment.

### DETAILED DESCRIPTION OF THE INVENTION

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the inventive closure device, as well as the axial ends of various component features. The term “proximal” is used in its conventional sense to refer to the end of the apparatus (or component thereof) that is closest to the operator during use of the apparatus. The term “distal” is used in its conventional sense to refer to the end of the apparatus (or component thereof) that is initially inserted into the patient, or that is closest to the patient.

According to some embodiments, the apical closure devices of the present disclosure may comprise one or more, and preferably all, of the following components: a support structure, preferably capable to be deformed for the positioning (reducing outer diameter, recovering a cylindrical shape); cover component, preferably covering partially or totally the outer surface of the support structure; and a plug for filling a cavity or lumen of the support structure, preferably capable of being punctured and passed through by a guidewire. In some embodiments, the plug may be optional, or it may be omitted if the cover component performs a desired sealing effect to block leakage through the access opening. The devices of the present invention allow for the positioning of the device over the wire for delivery to the access opening. The wire may pass through the closure device or outside the closure device (e.g., between the closure device and the periphery of the access opening in the ventricle wall). Some devices of the present invention may optionally allow the reintroduction of a guidewire in the ventricle at any moment after the closure of a ventricular port, opening or defect.

According to some embodiments, the closure device is preferably an over-the-wire device that permits the passage of a guidewire through the closure device at any time intra-operatively and/or after the procedure (optionally).

Exemplary Support structure

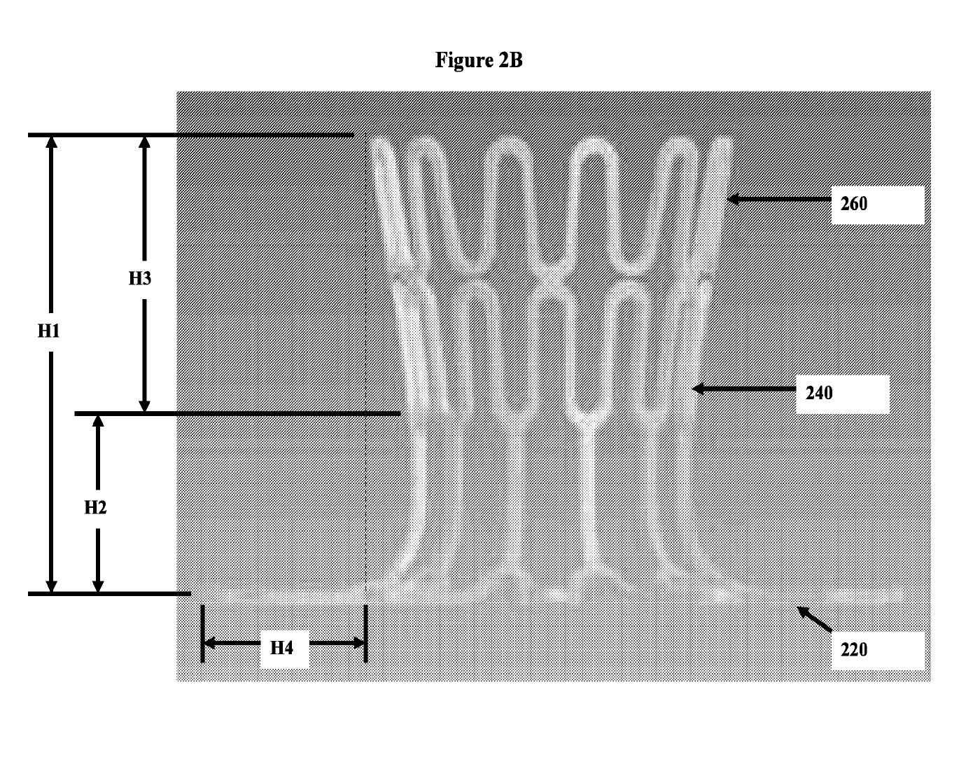

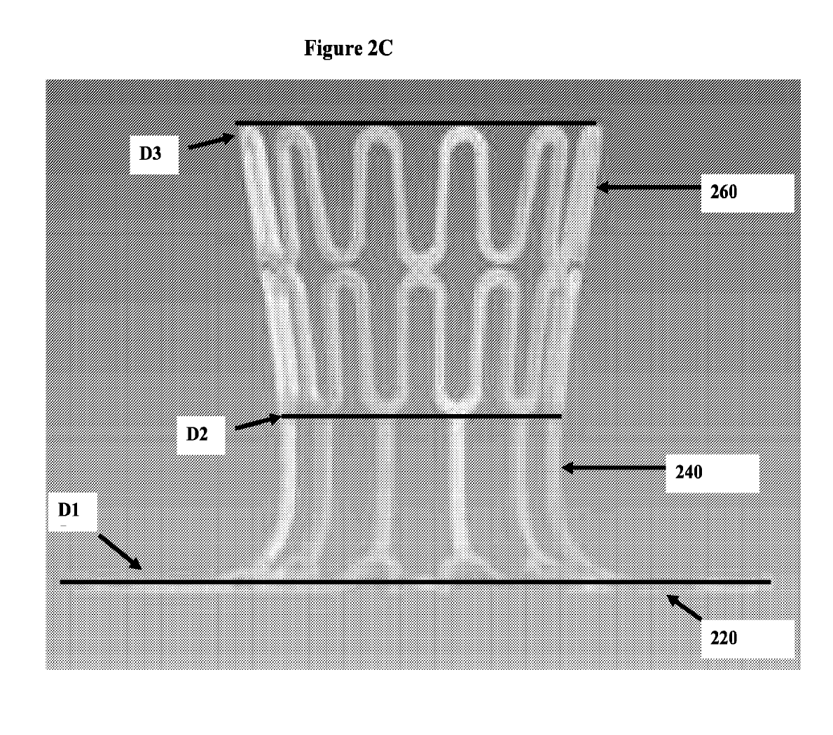

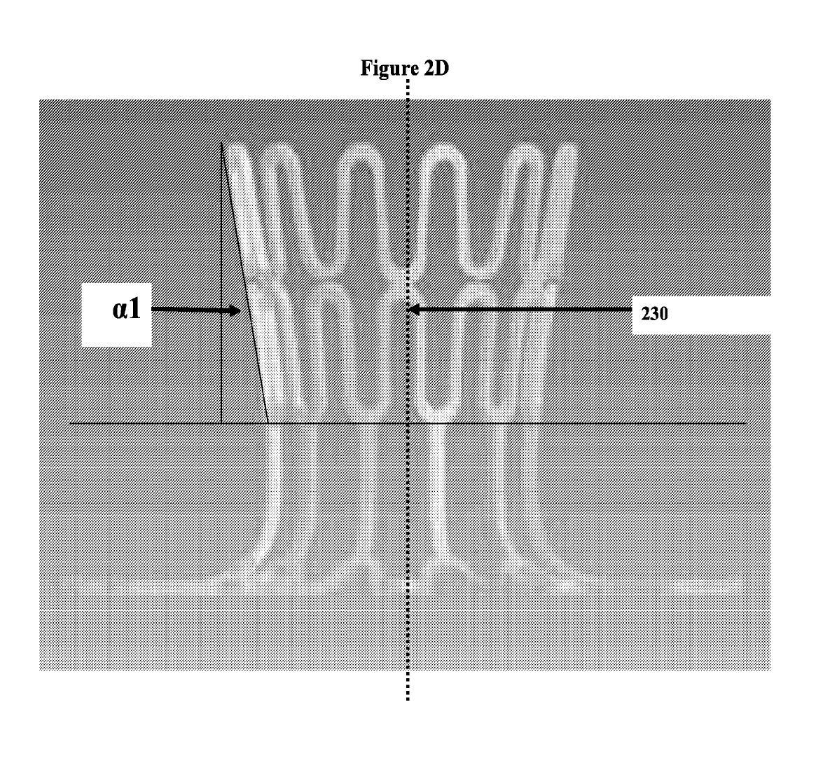

According to some embodiments, the support structure frame may be a tubular (e.g., cylindrical) body constructed from a plurality of serpentine wires (as generally shown in FIG. 2). In some embodiments, the wires are constructed of stainless steel and/or titanium. In some embodiments, the wires are constructed of Nitinol.

According to some embodiments, the closure device may be capable of expanding to a diameter exceeding the nominal diameter of the apical/surgical port/opening in order to cover the effect of laceration and weakening of the tissues due to the surgical opening. According to some embodiments, the support structure of the closure device may be capable of being deformed for positioning (e.g., compressed). The deformed state may be referred to as a delivery state for delivery to the access opening. This may be accomplished by reducing the outer diameter by a form of compression means familiar to one of skill in the art, and recovery of the cylindrical shape upon deployment. Thus, according to some embodiments, the cylindrical body portion is preferably expandable between a first compressed state (not shown) and a second expanded state (shown). According to some embodiments, the device is preferably self-expandable.

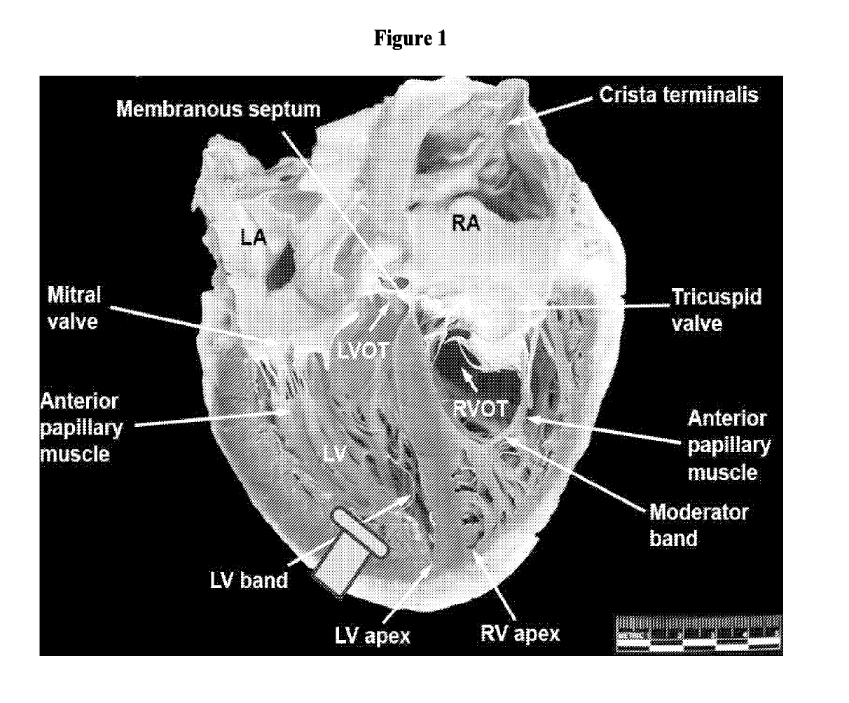

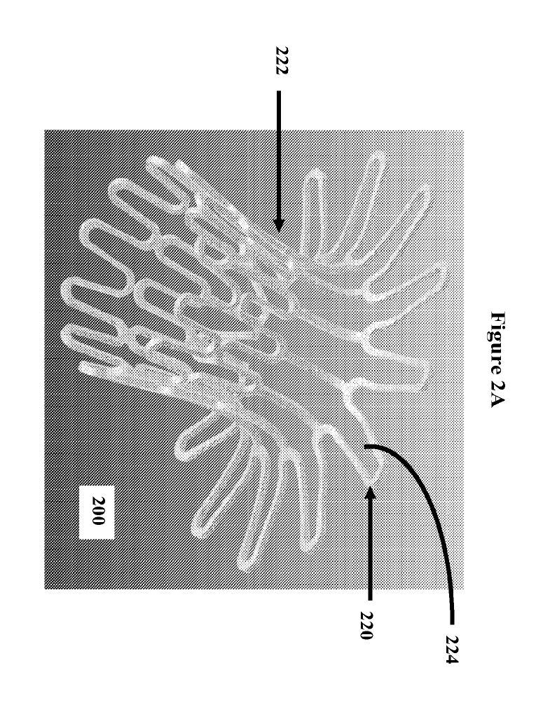

The supporting structure in its deployed state may have different diameters in order to self-secure on the cardiac wall. In particular, the portion of the closure device introduced in the ventricle (for example) preferably expands over the diameter of the access hole of the heart (e.g., access hole in the ventricular wall), in order to create a stop towards the inner ventricular pressure (FIG. 1), for example. A possible shape of the metallic structure is illustrated in FIGS. 2A-D. According to some embodiments, the supporting structure may be a meshed metallic structure (preferably self-expandable), capable of deformation for positioning in and/or delivery to the surgical opening (reducing outer diameter, recovering a deployed shape).

According to some embodiments, the support structure may be constructed from a mesh. The mesh may be constructed from, for example, wires (either a plurality of wires formed/welded together or a single wire), strips of shape memory material, such as nickel-titanium wire (e.g., Nitinol®). Nitinol, the nickel-titanium wire, when properly manufactured, exhibits elastic properties that allow for the wire to be manipulated (e.g., bent) by an operator and then returned to, substantially, the same shape the wire possessed prior to it being manipulated. For example, the wire returns to, substantially, the same shape the wire possessed prior to it being manipulated, for example, when the operator heats the wire or, alternatively, when the operator removes the forces applied to bend the wire.

The support structure, and sections thereof, may be formed, for example, by laser cutting a tube or single sheet of material (e.g., nitinol). For example, the support structure may be cut from a tube and then step-by-step expanded up to its final diameter by heat treatment on a mandrel. As another example, the support structure may be cut from a single sheet of material, and then subsequently rolled and welded to the desired diameter.

### Exemplary Dimensions

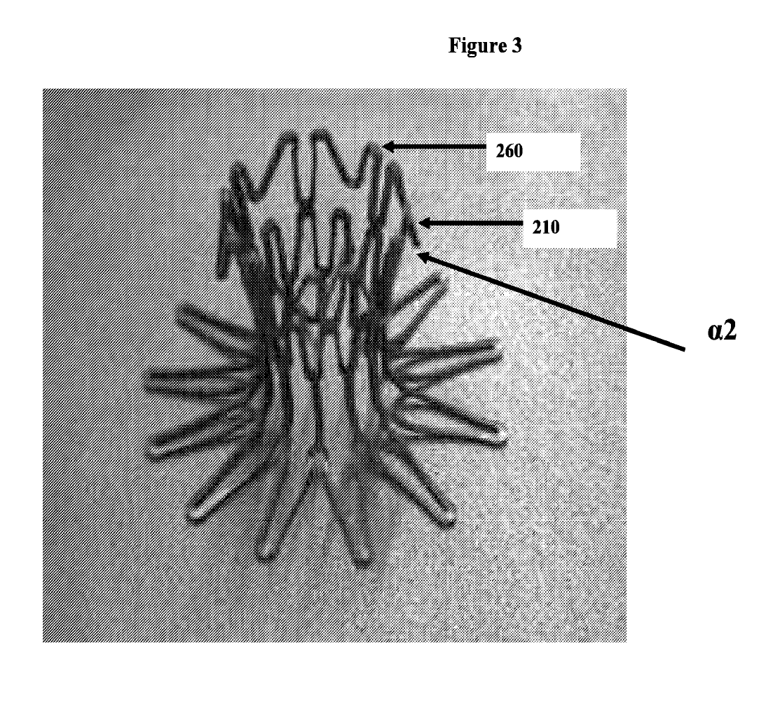

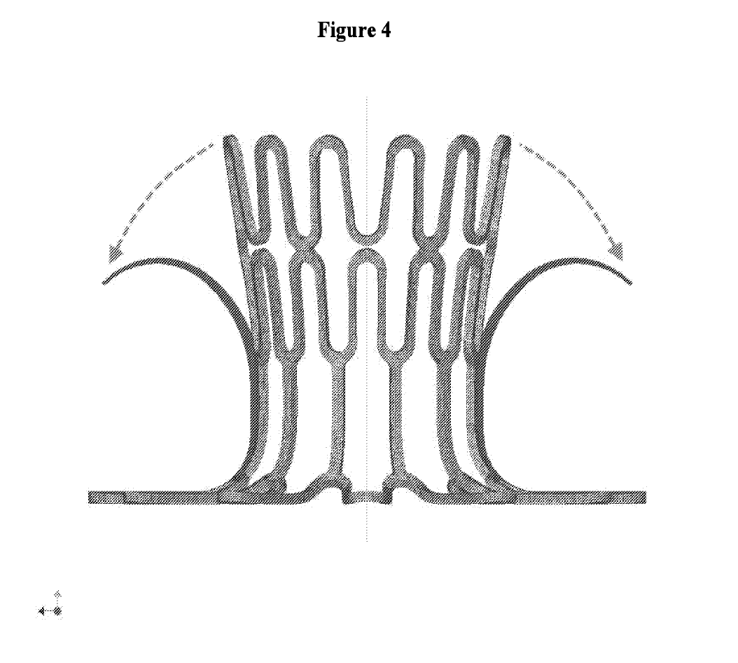

According to some embodiments, the length of the closure device may be self-adapting to the thickness of the ventricle wall. Accordingly, the proximal part of the closure device (the one towards the outside of the ventricle) is preferably configured to be flexible enough to deflect outside the cylindrical shape where the constraint of the ventricular wall is missing (pericardial space) (See e.g., FIG. 4).

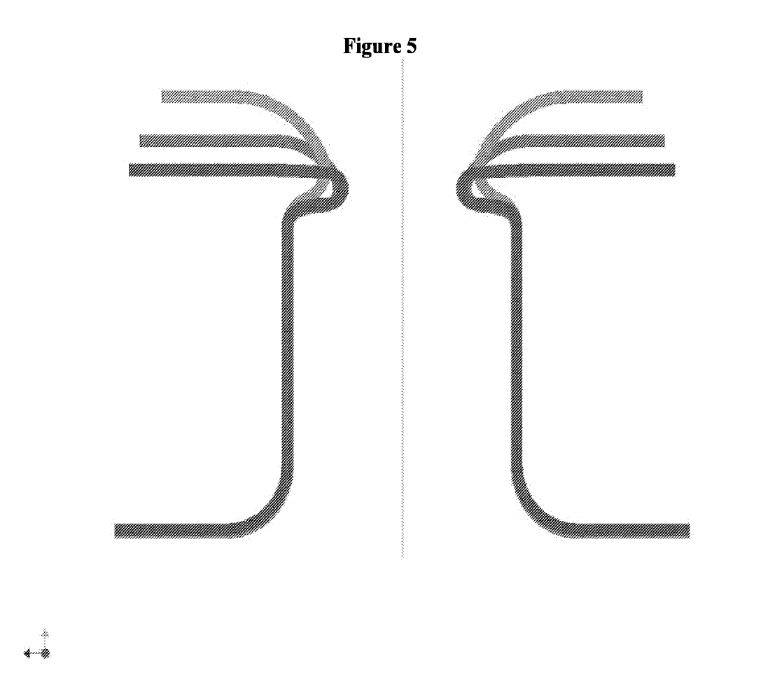

In another embodiment the length of the closure device is self-adapting to the thickness of the ventricle wall, due in part to its axial compliance See e.g., FIG. 5). FIG. 5 shows a flange that is created in correspondence of the proximal edge of the stent. The outer diameter of this flange can be smaller than the diameter of the flange positioned within the ventricle. Axial elastic joint (compliant coils) make the length of the device (i.e., the distance between the two flanges) variable and self-adaptable to the wall thickness.

According to some embodiments, the device may be capable of expanding to a diameter that exceeds the nominal diameter of the apical port/opening—an opening in the cardiac wall allowing ingress and egress of devices, which may also be referred to as a surgical opening, in order to cover any effect of laceration and weakening of the tissues due to the surgical opening.

The support structure may be configured such that, following implantation in the ventricle wall, the support structure does not protrude or project substantially from the surface of the ventricle wall, especially on the exterior surface of the ventricle wall (e.g., between about 0 mm and about 2 mm, and in some embodiments, between about 0.001 mm and about 2 mm). The support structure thus has a low profile height on the external side, reducing any risk of damage to the body surrounding the heart, for example, pericardium tissue.

The support structure preferably comprises a flange portion 220 (anchor portion) and a first/spigot portion 222. The first portion 222 may comprise a cylindrical portion 240, and an optional outwardly expanding conical section 260. That is the optional section 260 may be an extension of the cylindrical section 240, or may have a conical shape that is outwardly deflected at an angle (α1).

The flange portion 220 may be configured to fit/positioned substantially flat against the ventricle wall. A flat fit may avoid a substantial anchor portion projecting or protruding into the interior of the ventricle, and thereby avoid any risk of significant interference to blood flow in the ventricle and/or possibility of damage to internal tissue of the heart. To facilitate a flat fit, the flange portion 220 may have resilient conformability to adapt to the interior surface contour of the ventricle wall.

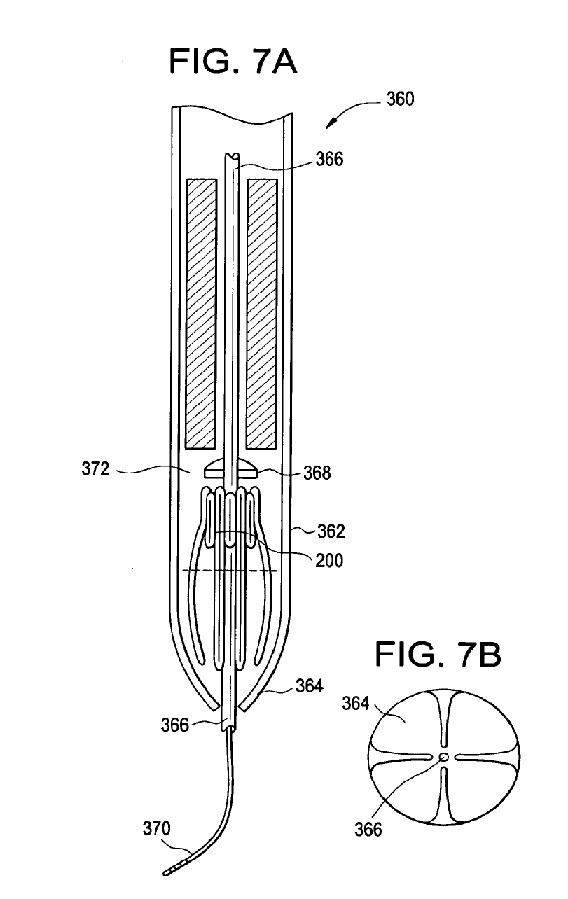

The flange portion 220 may be formed by a plurality of blades 224 (or blade-like elements) that extend radially outwardly from the first portion 222. The blades 224, or at least the tips thereof, may be substantially independent of one another. The blades 224 may provide high degree of conformability to adapt to the interior surface contour of the ventricle wall. Each blade 224 may fold or pivot independently of the other blades, so that the flange is not limited to a symmetrical or planar profile. For example, a portion of the flange portion engaging a relatively planar surface of the ventricle wall may extend generally perpendicularly from the first portion. A portion of the flange portion engaging a curved or concave surface region of the ventricle wall (especially near the apex) may curve to match, and fit flat against, the curved contour. Additionally or alternatively, the blades 224 may facilitate a relatively large flange body (in the deployed state) to be compressed to a relatively compact form (in the delivery state, e.g., in FIGS. 7-9). Each blade 224 may conveniently be defined by first and second struts the tips of which are coupled at the tips remote from the first portion 222. The blades 224 may have a skeletal form with clearances or apertures that, in use, may be covered by the cover component.

FIGS. 2A-D shows a support structure according to some embodiments. D1 represents the diameter of the flange of the support structure component in the expanded configuration 220. D2 represents a diameter of the cylindrical portion of the support structure component 240. According the preferred embodiments, the diameter of the cylindrical portion of the support structure component 240 exceeds the diameter of the opening in the cardiac wall.

The diameter of the flange section at D1 according to some embodiments is preferably between about 16 mm to about 40 mm (e.g., about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, and about 40 mm). This diameter D1 may be chosen depending to the size and shape of the access injury or depending on the size of the implantation device, or depending on the inner anatomy of the ventricle (e.g. left ventricle), or a combination of any two or more of the foregoing. Thus, the diameter of the flange in the expanded configuration D1 may be from between about 13 mm to about 50 mm, between about 15 mm to about 50 mm, from between about 15 mm to about 40 mm, from between about 15 mm to about 30 mm, from between about 15 mm to about 25 mm, from between about 15 mm to about 20 mm, from between about 20 mm to about 40 mm, from between about 24 mm to about 40 mm, from between about 26 mm to about 40 mm, from between about 28 mm to about 40 mm, from between about 30 mm to about 40 mm, from between about 32 mm to about 40 mm, from between about 34 mm to about 40 mm, from between about 36 mm to about 40 mm, from between about 38 mm to about 40 mm, from between about 22 mm to about 38 mm, from between about 22 mm to about 36 mm, from between about 22 mm to about 34 mm, from between about 22 mm to about 32 mm, from between about 22 mm to about 30 mm, from between about 22 mm to about 28 mm, from between about 24 mm to about 34 mm, from between about 25 mm to about 35 mm, or from between about 25 mm to about 30 mm.

The diameter of the cylindrical portion at D2 according to some embodiments is preferably between about 8 mm to about 20 mm (e.g., about 8 mm, about 9 mm, about 10 mm, about 11 mm, about 12 mm, about 13 mm, about 14 mm, about 15 mm, about 16 mm, about 17 mm, about 18 mm, about 19 mm, and about 20 mm). This diameter D2 may be adjusted depending to the size and shape of the access injury or depending on the size of the implantation device. Preferably, the diameter at D2 exceeds the diameter of the opening in the cardiac wall. According the some embodiments, the diameter D2 may be from between about 15 mm to about 50 mm, from between about 15 mm to about 40 mm, from between about 20 mm to about 40 mm, from between about 24 mm to about 40 mm, from between about 26 mm to about 40 mm, from between about 28 mm to about 40 mm, from between about 30 mm to about 40 mm, from between about 32 mm to about 40 mm, from between about 34 mm to about 40 mm, from between about 36 mm to about 40 mm, from between about 38 mm to about 40 mm, from between about 22 mm to about 38 mm, from between about 22 mm to about 36 mm, from between about 22 mm to about 34 mm, from between about 22 mm to about 32 mm, from between about 22 mm to about 30 mm, from between about 22 mm to about 28 mm, from between about 24 mm to about 34 mm, from between about 25 mm to about 35 mm, or from between about 25 mm to about 30 mm.

H1 represents the total maximum length through the thickness of the cardiac/ventricle wall (for example). H1, according to some embodiments, may be defined by the axial distance between the planes of the diameters D1 and D3 in the expanded configuration, or the combined lengths of the cylindrical portion (e.g., H2) of the support structure 240 and the extended portion (e.g. H3) of the support structure 260 in the expanded configuration. D3 is a function of the diameter D2, heights H1 and H2, and the angle α1, according to some embodiments. Preferably, H1 is between about 10 to about 25 mm (e.g., about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, and about 25 mm). For example, the length H1 may range from about 5 to about 25 mm, about 5 to about 24 mm, about 5 to about 20 mm, about 5 to about 10 mm, about 10 to about 25 mm, about 10 to about 24 mm, about 10 to about 20 mm, about 10 to about 15 mm, about 15 to about 25 mm, about 15 to about 24 mm, about 15 to about 20 mm, about 20 to about 25 mm, and about 20 to about 24 mm.

H2 represents the length of the cylindrical portion according to some embodiments. H2 represents the axial distance between the planes of the diameters D1 and D2 in the expanded configuration, or the length of the cylindrical portion of the support structure 240. In some embodiments, H2 is preferably between about 5 to about 20 mm (e.g., about 5 mm, about 6 mm, about 7 mm, about 8 mm, about 9 mm, about 10 mm, about 11 mm, about 12 mm, about 13 mm, about 14 mm, about 15 mm, about 16 mm, and about 20 mm). The length H2 may be adjusted depending on the intended application of the support structure. According to some embodiments, the length of H2 may range from about 6 to about 16 mm, about 5 to about 20 mm, about 5 to about 15 mm, about 5 to about 16 mm, about 5 to about 10 mm, about 10 to about 20 mm, about 10 to about 15 mm, about 10 to about 16 mm, and about 10 to about 12 mm.

H3 represents the axial distance between the planes of the diameters D2 and D3 in the expanded configuration, or the length of the portion of the support structure in the expanded configuration that may be deflected in an outward direction to adjust the length of the apical device to the thickness of the ventricle wall 260 according to some embodiments. Preferably, H3 is between about 3 mm to about 10 mm (e.g., about 3 mm, about 4 mm, about 5 mm, about 6 mm, about 7 mm, about 8 mm, about 9 mm, and about 10 mm). In some embodiments, the length of H3 and degree of outward deflection (α1) may be adjusted to the size and shape of the access injury or on the size of the implantation device. For example, H3 may range from about 3 mm to about 10 mm, about 3 to about 15 mm, about 4 mm to about 10 mm, about 4 to about 15 mm, about 4 to about 9 mm, about 4 to about 8 mm, about 4 to about 7 mm, about 4 to about 6 mm, about 5 to about 10 mm, about 7 to about 10 mm, about 7 to about 12 mm, about 7 to about 15 mm, about 10 to about 13 mm, about 5 to about 15 mm, about 5 to about 8 mm.

1-14 represents the dimension of the flange of the support structure 220 in the expanded configuration according to some embodiments. Preferably, the length of the flange 220 (H4) is greater then about 2 mm. According to some embodiments, H4 is between about 3 to about 50 mm (e.g., about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 20 mm, about 22 mm, about 24 mm, about 25 mm, about 26 mm, about 28 mm, about 30 mm, about 32 mm, about 34 mm, about 36 mm, about 38 mm, about 40 mm, about 42 mm, about 44 mm, about 45 mm, about 46 mm, about 48 mm, and about 50 mm). According to some embodiments, H4 may range from about 3 to about 40 mm, about 3 to about 30 mm, about 3 to about 20 mm, about 3 to about 10 mm, about 3 to about 8 mm, about 3 to about 6 mm, about 3 to about 5 mm, 5 to about 40 mm, about 5 to about 30 mm, about 5 to about 20 mm, about 5 to about 10 mm, about 10 to about 50 mm, about 10 to about 40 mm, about 10 to about 30 mm, about 10 to about 20 mm, about 15 to about 50 mm, about 15 to about 40 mm, or about 15 to about 30 mm. In some embodiments, the range for H4 may have a minimum as any of the aforementioned, and a maximum of about 15 mm.

The α1 angle defines the outward deflection of an optional third conical section of the support structure according to some embodiments. The α1 angle is preferably between from about 0 degrees to about 50 degrees with respect to the support structure axis (e.g., about 5 degrees, about 10 degrees, about 15 degrees, about 20 degrees, about 25 degrees, about 30 degrees, about 35 degrees, about 40 degrees, about 45 degrees, and about 50 degrees. According to some embodiments, the α1 angle is between from about 5 degrees to about 45 degrees, between from about 5 degrees to about 40 degrees, between from about 5 degrees to about 30 degrees, between from about 5 degrees to about 25 degrees, between from about 5 degrees to about 20 degrees, between from about 5 degrees to about 15 degrees, between from about 5 degrees to about 10 degrees, between from about 10 degrees to about 15 degrees, between from about 8 degrees to about 12 degrees, between from about 10 degrees to about 20 degrees, between from about 10 degrees to about 25 degrees, between from about 10 degrees to about 30 degrees, between from about 15 degrees to about 20 degrees, or between from about 15 degrees to about 25 degrees.

The support structure component illustrated in FIG. 3 includes some additional features, mainly one or more anchoring elements 210 in support structure. According to some embodiments, such protrusions may be formed generally in the shape of a bent, or curved angled member (e.g., an “L” or “J” like shape). In some embodiments, such attachment elements may be a hook (e.g., a “J” like shape). According to some embodiments, the device may have some protrusions 210 in order to improve the anchorage to the ventricle wall. According to preferred embodiments, the device may have some protrusions 210 on the lateral side in order to improve the anchorage to the ventricle wall.

According to preferred embodiments, protrusions slant downwards from the distal end of the device at an angle α2 with respect to the support structure axis. The α2 angle is preferably between from about 10 degrees to about 60 degrees with respect to the support structure axis (also, with respect to the longitudinal axis 230 of the closure device), and optionally at least 30 degrees. Thus, according to some embodiments of the present disclosure, the α2 angle is preferably about 10 degrees, about 15 degrees, about 20 degrees, about 25 degrees, about 30 degrees, about 35 degrees, about 40 degrees, about 45 degrees, about 50 degrees, about 55 degrees, or about 60 degrees. According to some embodiments, the α2 angle is between from about 30 degrees to about 55 degrees, between from about 30 degrees to about 50 degrees, between from about 30 degrees to about 45 degrees, between from about 30 degrees to about 40 degrees, between from about 30 degrees to about 35 degrees, between from about 40 degrees to about 60 degrees, between from about 40 degrees to about 50 degrees, between from about 45 degrees to about 60 degrees, or between from about 45 degrees to about 55 degrees.

The length of the protrusions 210 may vary. According to some embodiments, the length of the protrusions is about 50% of the length of H3. Thus, according to some embodiment of the present invention, the length of the protrusions is preferably about 45% of the length of H3, about 40% of the length of H3, about 35% of the length of H3, about 33% of the length of H3, about 30% of the length of H3, about 25% of the length of H3, about 20% of the length of H3, about 15% of the length of H3, or about 10% of the length of H3.

The number of protrusions or anchors 210 may also vary. According to some embodiments, the support structure comprises 2 to 20 (e.g., 2, 3, 4, 5, 6, 7, 8, 9, 10, 12, 14, 16, 18, or 20) protrusions or anchors 210.

### Cover

In some embodiments, the cover (which may also be referred to as the cover component) is preferably designed to be flexible, biocompatible, and non-thrombogenic. According to some embodiments, the function of the cover component is to insure the adequate sealing of the device against the risk of bleeding due to the inner cardiac/ventricular pressure.

According to some embodiments, the cover component may be made of biological tissues and/or artificial materials. According to some embodiments, the cover component is made of cover material that is a biocompatible material. This includes, but is not limited to, polymeric rubber, artificial fabric, or bovine, porcine or human tissue that is chemically treated to minimize the likelihood of rejection by the patient's immune system, or a combination of these materials. Synthetic biocompatible materials such as polytetrafluoroethylene, polyester fabric (e.g., Dacron®), double polyester fabric, woven polyester (e.g., polyethylene terepthalate), polyurethane, nitinol or other alloy/metal foil sheet material and the like may be used. According to some embodiments, the cover component is made from fresh, cryopreserved or glutaraldehyde fixed allografts or xenografts. According to some embodiments, the cover component is made from mammal pericardium tissue, particularly juvenile-age animal pericardium tissue. According to some embodiments, the covering material is comprised of a polyester material, such as a single ply polyester material or polyethylene terephthalate (PET).

According to some embodiments, the cover material covers partially or totally the outer surface of the support structure. According to some embodiments, the cover material covers all, or substantially all, of the support structure outer surface. According to some embodiments, the cover material may be configured to improve endothelialisation.

According to preferred embodiments, the cover material cover at least about 30% of the outer surface of the support structure 200. This includes, for example, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, and at least about 99% of the outer surface of the support structure 200.

According to some embodiments, the covering material may cover at least a portion—for example, either a minor portion (e.g., less than about 20% coverage), a substantial portion (e.g., about 50-90% coverage), or all (e.g., 90%+) of the support structure) of the outer surface of the support structure. The cover may be sutured to the inner surface of the support structure. In some embodiments, it will be desirable to suture the cover material on the outer surface of the support structure. For example, the cover material may wrap the support structure, partially or completely.

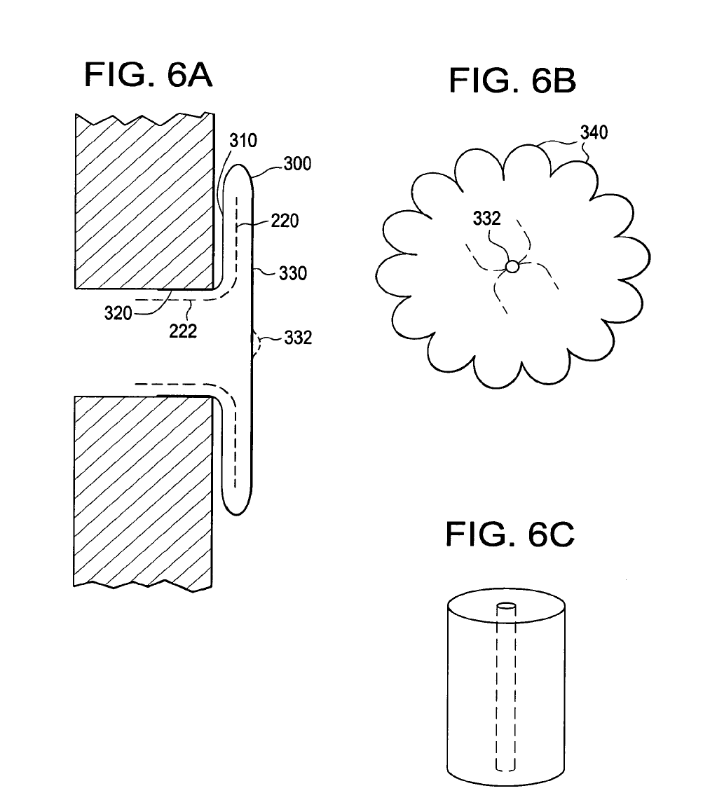

FIG. 6 illustrates one example of a cover component 300. The cover component optionally includes an annular (or other closed-loop shape) washer portion 310 for forming a seal between the flange portion 220 and the ventricle wall. The cover component optionally includes a tubular collar portion 320 for sealing between the first portion 222 and the periphery of the access opening bore. The tubular collar portion 320 may be of about the same length as, or shorter than, the first portion 222. The cover component 300 optionally includes a fascia portion 330 for the face of the flange portion 220 facing into the ventricle. The fascia portion 330 may extend over the entire face of the flange portion. The fascia portion 330 may additionally extend over the central lumen of the support structure to occlude the lumen. The fascia portion 330 may optionally comprise or carry a self-closing valve or aperture 332 through which a guidewire may pass. The valve 332 may comprise one or more flaps or leaflets. The valve 332 may be integrated as part of the cover component 300. The valve 332 may be made of the same material as the cover component.

Referring to FIG. 6b, the periphery of the cover component 300 may have a shape formed as a sequence of petals 340. The petals 340 may be aligned with, or follow the contour of, the blades of the support structure. The presence of petals avoids excess material in the troughs between adjacent petals, thereby facilitating crimping or compressing of the closure device.

The cover component 300 may be made of a single piece of material, or it may comprise a plurality of pieces attached together (e.g., by sutures).

### Plug

When used, the plug component of the closure devices (e.g., ventricular wall closure devices) of the present invention fills the inner lumen of the support structure 200. According to some embodiments, the plug component is a polymeric plug, or a foldable haemostatic valve, or foam, or polymeric sponge filling, or a combination of any of the foregoing. Preferably, the plug is made up of a material or mixture of material that is capable of being punctured and passed through by a guidewire. For example, the plug may be a self-sealing silicone plug that fills the inner lumen of the support structure.

According to some embodiments, the plug component is a haemostatic valve or be constructed of self-sealing silicone plug. Any other sealing material that can be used as a plug is contemplated within the invention. According to some embodiments, the plug seals the lumen of the support structure against hydrostatic pressure, but preferably allows passage of a needle, probe, balloon catheter or any tissue operative instrument known to those in the art, by way of a slit, for operation. The plug seal resiliently closes after removal of instrument.

In some embodiments, the plug can be pre-mounted on the device, i.e., it is deployed together with the device, or can be inserted after the deployment of the device. In the latter case, the sealing effect is totally in charge of the device cover. In both case the plug could be made removable, if necessary.

By way of example, FIG. 6C illustrates one form of plug 350 suitable for filling the lumen of the support structure 200. The plug may be oversized, and compressed to provide a resilient form fit within the support structure 200.

### Deployment/Delivery System

The closure devices of the present invention are preferably deployed to seal an open ing in a cardiac wall, preferably a cardiac/ventricular wall. Sealing is possible where a reaction force is generated at an interface between the closure device (e.g. metallic mesh) and the ventricle wall. Therefore, sealing may optionally be effected between the first portion 222 and the interior bore surface of the access opening. That is, the closure device applies a radial force against the wall of the ventricle, which may be obtained and controlled (e.g., selecting appropriate size, shape, material, etc. of the closure device). Preferably, the device is capable of expanding to a diameter exceeding the nominal diameter of the opening for closure in order to cover the effect of laceration and weakening of the tissues due to the surgical opening.

Additionally or alternatively, sealing may optionally be effected between the flange portion 220 and the face of the ventricle wall confronted by the flange portion. An axial force may maintain a seal reaction force. For example, the closure device 200 may be deployed in a state of tension. Alternatively, the length of the device may be (e.g. resiliently) self-adapting to the thickness of the ventricle wall. That is, the proximal part of the device (the one towards the outside of the ventricle) is flexible enough to reverse outside the cylindrical shape where the constraint of the cardiac wall (e.g., ventricular wall) is missing (pericardial space) or the length of the device self-adapt to the thickness of the wall.

According to some embodiments, the device may be self-expandable. The device, alternatively, could be expanded by heating the shape memory material once the device is located in a desirable location in the heart. The device could warm due to contact with, for example, heart tissue or blood of the patient.

According to some embodiments, the device can be positioned by a dedicated delivery system. According to some embodiments, the closure devices of the present disclosure may be designed to be implanted with the use of an introducer device (e.g., a catheter delivery device).

For deployment, the apical closure device may be inserted through an access opening (e.g., surgical opening) of a cardiac wall, preferably a ventricular wall, and more preferably, the wall of the left ventricle. Preferably, the closure device is advanced into the heart in the collapsed or folded configuration. The device may be then be expanded and positioned by removal of the forces that maintain the devices in the collapsed configuration. For example, the closure device and be expanded and positioned by sliding device out of a sheath of a delivery catheter, which first causes expansion of a distal end of the apical closure device followed by expansion of the proximal end of the apical closure device as the device is slid out of the sheath. In this manner, the apical closure device is released from the sheath in order to cause full expansion of the apical closure device. In some embodiments, the apical closure device may be recaptured prior to its full expansion by sliding the sheath in the opposite direction.

According to some embodiments, an introducer, such as an introducer generally used during cardiac surgical procedures to keep a surgical opening or port “open” and rapidly accessible, can position the device. For example, the closure device according to some embodiments may be slid within the introducer until the flange located at the distal end of the closure device (e.g., distal flange/blades/petals) open inside the heart (e.g., ventricle). The introducer may then be slowly withdrawn until the flange/blades/petals engage the inner wall of the ventricle. Upon final removal of the introducer from the cardiac wall opening causes the complete release of the closure device and its positioning across the cardiac wall. Similar delivery procedures can be followed also with a dedicated delivery system

According to some embodiments, the closure device may be capable of being compressed and loaded into an introducing catheter. Subsequent to insertion of the introducing catheter into the heart of a patient and locating the introducing catheter in a desirable location, an operator deploys the device from the introducing catheter. The device then expands because the introducing catheter no longer applies a constraining force to the device. In another aspect, the device self-expands once it is withdrawn from the introducing catheter. Such catheter delivery systems can be found, for example, in WO 2008/028569, herein incorporated by reference.

According to some embodiments, the device may be an over-the-wire device. For example, the device is designed to allow the passage of a guidewire through the device at any time, intra-operatively or after some time after the surgical procedure.

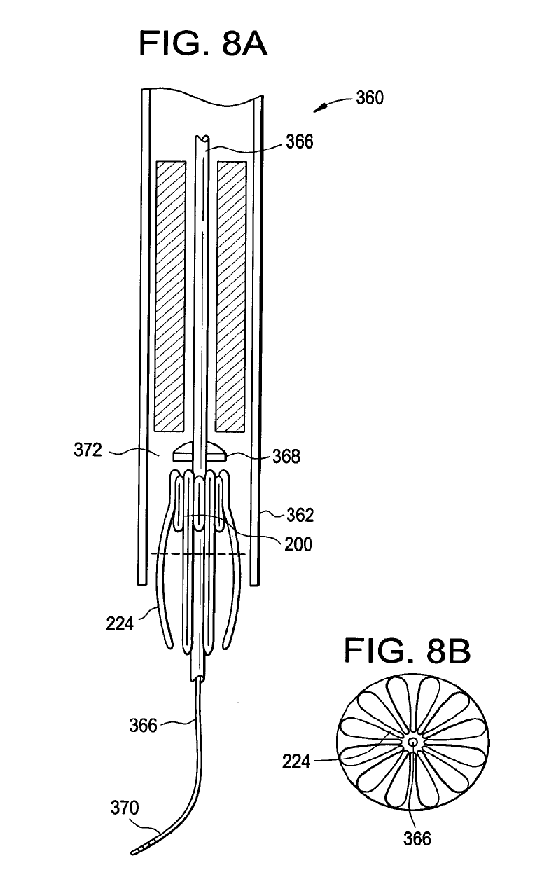

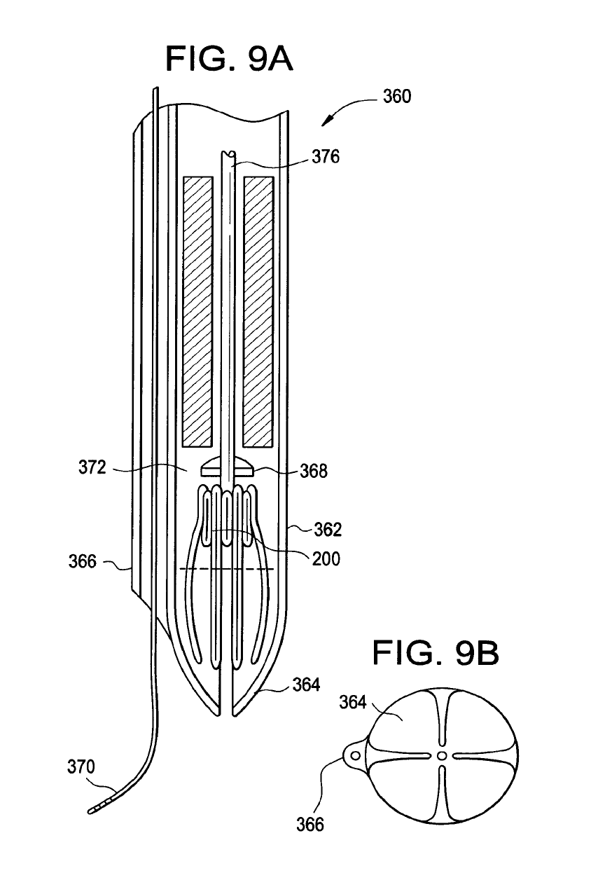

By way of example, FIGS. 7-9 illustrate different examples of over-the-wire delivery devices 360. The devices generally include a sheath 362 defining a compartment 372 for receiving and constraining at least a portion of the closure device 200, such that the closure device is maintained in its delivery or compressed state. In FIGS. 7 and 9, the sheath substantially entirely surrounds the entire length of the closure device. The tip 364 of the sheath is segmented, for example, as flexible leaves delimited by axially extending slots. The segments or leaves curl inwardly to define a rounded or tapered tip of the delivery system to allow the delivery device to advance non-traumatically within the body to the access opening. The flexibility of the leaves or segments allows the closure device to emerge from the sheath at implantation as explained below. In the example of FIG. 8, the sheath 362 does not surround a distal end of the closure device 200. Instead, the distal end of the closure device 200 is exposed, and defines its own tapered tip shape. The tapered tip shape is defined by distal portions of the closure device that, in the delivery state, curl or curve distally inwardly. The distal portion may be formed by the blades 224.

The delivery devices 360 further comprise a guidewire lumen 366 for receiving a pre-disposed guidewire 370, and allowing the delivery device to be guided to the site of implantation. In Figures. 7 and 8, the guidewire lumen 366 is generally centrally located along the axis of the sheath 362, and passes through the closure device 200 (e.g., through the plug and/or cover component). In use, in order to deploy the closure device 200, the sheath 362 is pulled proximally, exposing and deploying the closure device progressively from its distal end. An abutment shoulder 368 maintains the closure device in position as the sheath is pulled proximally, to ensure that the closure device releases cleanly from the sheath 362 despite any friction between the sheath 362 and closure device 200. Thereafter, the guidewire is withdrawn, and the aperture or valve within the closure device seals itself following the withdrawal of the guidewire.

In the example of FIG. 9, the guidewire lumen 366 is arranged outside the compartment containing the closure device 200, such that the guidewire 370 does not pass through the closure device 200. Such an arrangement still permits the delivery device to be guided to the site of implantation using a guidewire, but avoids having to provide a self-sealing valve or aperture in the cover component and/or optional plug of the closure device. The shoulder 368 may instead be mounted on a support shaft 376 that does not pass through the closure device 200. When the closure device 200 is released and deployed from the delivery device 360, the guidewire 370 becomes accommodated to one side of the closure device 200 fitting at the serpentine interface between the closure device 200 and the ventricle wall. For example, the guidewire may bend or fold to comply with the serpentine shape defined by the profile of the support structure. The guidewire may be aligned with a trough between adjacent petals 340 to reduce the length of serpentine deformation in the guidewire. Following implantation, the flexibility of the guidewire allows the guidewire to be withdrawn along the serpentine path by pulling the guidewire proximally. Withdrawal of the guidewire from the contact interface allows the closure device 200 to seat fully against the ventricle wall.

Some embodiments of the present disclosure provides of methods for sealing ventricular ports, surgical openings in cardiac walls, and cardiac wall defects, and may comprise delivering an implantable expandable closure device into the ventricle via a catheter. The expandable device is then expanded to assume the size and shape of the port, opening or defect in the wall of the left ventricle and preferably anchored to the wall of the left ventricle.

### Medical Uses

According to some embodiments, the devices of the present methods are used in conjunction with medical procedures to correct defects or diseases of the heart (e.g., septal defects, valve replacement) via a transapical delivery (i.e., direct access, through the wall of the heart access). For example, in a transapical delivery, the patient may receive a small puncture in the chest cavity where the operator could, for example, access the apex of the heart similar to a ventricular assist device implantation. Once access is gained to the left ventricle, the aortic and mitral valves are a direct pathway for implantation of the replacement valve. In this case, the aortic valve would be delivered with the flow path in the same direction as the catheter. For the mitral valve, the flow path would be against the direction of implantation. In this manner, medical procedures using transapical access allows for the devices to be placed in a less invasive surgical procedure. For example, transapical access permits a beating-heart procedure, but limits the access incision area. Through the apex of the heart a tube may be inserted to introduce the device to the aortic valve from an antegrade approach.

According to some embodiments, the devices of the present methods are used in conjunction with medical procedures for replacing a worn or diseased valve comprising transapically implanting a replacement valve.

Any and all references to publications or other documents, including but not limited to, patents, patent applications, articles, webpages, books, etc., presented in the present application, are herein incorporated by reference in their entireties.

Although a few variations have been described in detail above, other modifications are possible. For example, any logic disclosed in the present disclosure does not require the particular order shown, or sequential order, to achieve desirable results. Other embodiments are possible, some of which, are within the scope of the following claims.

1. A closure device implantable via an access opening in the ventricle wall of a heart and operative to close the access opening, the device comprising:
a support structure collapsible to a delivery state for implantation via the access opening, the support structure being self-expandable when released to expand to a deployed state in which the support structure defines at least (i) a first portion configured for positioning within the access opening, and (ii) a flange portion coupled to the first portion and configured for positioning substantially flat against the ventricle wall to prevent migration of the closure device in at least one direction following implantation; and
a cover to at least partially cover the outer surface of the support structure.

2. The closure device of claim 1, wherein the flange portion is coupled to a distal portion of the first portion for bearing against the interior surface of the ventricle wall to prevent migration of the closure device in a direction out of the ventricle.

3. The closure device of claim 1, further comprising at least one protrusion to engage the ventricle wall for preventing migration of the closure device into the ventricle, the protrusion being configured to project not more than between about 0.001 mm and about 2 mm from the exterior surface of the ventricle wall.

4. The closure device of claim 3, wherein the protrusion is configured to at least one of: (i) engage a portion of the ventricle wall inside the access opening; (ii) conform to the exterior surface of the ventricle wall; and (iii) comprise the proximal flange coupled to the first portion by a resilient coupling.

5. The closure device of claim 1, wherein the flange portion is coupled to a proximal portion of the first portion for bearing against the exterior surface of the ventricle wall.

6.-7. (canceled)

8. The closure device of claim 1, wherein the cover comprises or carries a substantially self closing aperture for passing a guidewire therethrough.

9. (canceled)

10. A closure device implantable via an access opening in the ventricle wall of a heart and operative to close the access opening, the device comprising:
a support structure collapsible to a delivery state for implantation via the access opening, the support structure being self-expandable when released to expand to a deployed state in which the support structure defines at least (i) a first portion configured for positioning within the access opening, and (ii) an anchor portion coupled to the first portion and configured for bearing against the ventricle wall to prevent migration of the closure device in at least one direction following implantation, the anchor portion comprising a plurality of radially outwardly projecting independent spokes; and
a cover for at least partially covering the outer surface of the support structure.

11. (canceled)

12. The closure device of claim 10, wherein each spoke comprises first and second struts coupled at their tips remote from the first portion.

13. The closure device of claim 10, wherein the cover includes a portion having a shape formed as a sequence of petals aligned with the spokes.

14. A device for closing an access opening in the ventricle wall of a heart comprising:
a support structure having a lumen;
a cover for at least partially covering the outer surface of the support structure; and
a plug for substantially filling the lumen of the support structure.

15. The closure device of claim 14, wherein the support structure comprises a flange portion, a cylindrical portion, and an optional outwardly expanding conical portion.

16. The closure device of claim 15, wherein the diameter of the cylindrical portion exceeds the apical opening diameter.

17. The closure device of claim 15, wherein the diameter of the cylindrical portion is between about 8 mm to about 18 mm.

18. The closure device of claim 15, wherein the length of the cylindrical portion is between about 10 mm to about 25 mm.

19. The closure device of claim 15, wherein the length of the cylindrical portion and the optional outwardly expanding conical portion is between about 10 mm to about 25 mm.

20. The closure device of claim 15, wherein the length of the cylindrical portion is from about 6 to about 16 mm and the length of the outwardly expanding conical portion is from about 4 mm to about 10 mm.

21. The closure device of claim 15, wherein the angle that defines the outward deflection of an optional third conical portion of the support structure is between about 0° to 20°.

22.-23. (canceled)

24. The closure device of claim 14, wherein the plug is capable of being punctured and passed through by a guidewire.

25.-27. (canceled)

28. The closure device of claim 1, wherein the support structure is a self-expandable meshed metallic support structure.

29. The closure device claim 1, wherein the diameter of the flange portion is between about 13 mm to about 40 mm.

30.-39. (canceled)
